(19) United States
(12) Patent Application Publication
(10) Pub. No.: US 2021/0383936 A1
(43) Pub. Date: Dec. 9, 2021

(54) Network Architecture In Psychopathological Symptomology

(71) Applicant: X Development LLC, Mountain View, CA (US)

(72) Inventors: Vladimir Miskovic, Binghamton, NY (US); Katherine Elise Link, Palo Alto, CA (US); Mustafa Ispir, Mountain View, CA (US); Pramod Gupta, Mountain View, CA (US)

(21) Appl. No.: 16/891,757

(22) Filed: Jun. 3, 2020

(51) Int. Cl. G16H 70/60; G16H 20/70; G16H 10/20; G16H 40/63; G16H 20/30; G06F 17/18; G06F 16/28; G06N 3/04; A61B 5/00

(52) U.S. Cl. CPC G16H 70/60; G16H 20/70; G16H 10/20; G16H 40/63; G16H 20/30; A61B 5/7246; G06F 16/285; G06N 3/04; A61B 5/6813; A61B 5/743; G06F 17/18

(57) ABSTRACT

Methods, systems, and apparatus, including computer programs encoded on a computer storage medium, for receiving physiological data of a patient, obtaining ecological momentary assessment (EMA) data by sending an EMA data prompt, and receiving patient input responsive to the EMA data prompt; and generating, based on the EMA data and the physiological data, a graphical representation of the patient's idiomatic psychopathology symptom network as a symptom network graph.

### TECHNICAL FIELD

This disclosure generally relates to a system of tracking an individual patient's psychopathology through applied graph theory.

### BACKGROUND

Current diagnostic psychology relies on administration of questionnaires to generate static snapshots of a patient's psychological profile across a range of subjective symptoms. These questionnaires can be directed towards known psychiatric disorders, like major depressive disorder (MDD) or anxiety. The questionnaires can provide an output score summed across the individual responses of the questions contained in a questionnaire. The use of a sum score can mask changes within the individual responses, particularly as it relates to beginning or changing pharmacological drug use. Within a single questionnaire, the effects of a pharmacological drug can be masked by the sum score to provide misdirecting output information.

### SUMMARY

This disclosure generally relates to a system of tracking an individual patient's psychopathology through applied graph theory. The system uses wearables and prompted responses to collect objective (e.g. heart rate, sleep patterns) and subjective (e.g. responses to sense of self, mood, appetite) measures through time. Once a baseline has been collected (e.g. over several days or weeks), the patient is continuously monitored (e.g. over several months or years) to reveal underlying variability and response to treatments (e.g. prescribed medication). The method can provide a clinician means to determine causal symptom relationships which can be targeted for treatment. This would allow greater efficiency, specificity, and control in developing a psychological or pharmaceutical treatment plan. Further, when reviewing a patient's unique symptom network a clinician may choose the level of detail they are interested in from the output from high level symptom causal relationships to granular individual symptom changes through time in response to treatment.

In particular, pharmaceutical treatment outcomes can be masked by present psychopathology assessments due to their reliance on summarized scores. By tracking a patient's idiomatic psychopathology through time, response to treatments can be tracked on a symptom by symptom basis and inter-symptom correlation monitored for changes. This could allow faster and more specific treatment outcomes to be tracked by a clinician and targeted treatments for specific central symptoms to allow efficiency in treatment.

In general, innovative aspects of the subject matter described in this specification can be embodied in methods that include the actions of receiving, from a wearable computing device, physiological data of a patient, the physiological data being received over a period of time; obtaining, at predetermined time intervals within the period of time from the patient, ecological momentary assessment (EMA) data by, at each time interval: sending, for presentation on a computing device associated with the patient, an EMA data prompt, and receiving, from the computing device, patient input responsive to the EMA data prompt; and generating, based on the EMA data and the physiological data, a graphical representation of the patient's idiomatic psychopathology symptom network as a symptom network graph, the graph includes symptom nodes connected by edges, the symptom nodes representing individual psychopathologic symptoms present in the individual and the edges representing correlations between different symptom nodes indicated by the EMA data and the physiological data. Other implementations of this aspect include corresponding systems, apparatus, and computer programs, configured to perform the actions of the methods, encoded on computer storage devices.

These and other implementations can each optionally include one or more of the following features.

In some implementations, a characteristic of the edges represents strength of correlation between symptom nodes.

In some implementations, generating the symptom network graph further includes applying the physiological data and the EMA data as input to a clustering model to obtain correlation data indicating correlations between symptoms measured by the physiological data and the EMA data; and generating the symptom network graph based on the correlation data.

In some implementations, the clustering model is one of a community detection algorithm, a k-mean clustering algorithm, a mean-shift clustering algorithm, an expectation-maximization clustering algorithm, or an agglomerative hierarchical clustering algorithm.

In some implementations, generating the symptom network graph further includes applying the physiological data and the EMA data as input to an auto-regression model to obtain causality data indicating casuals relationships between symptoms measured by the physiological data and the EMA data; and generating the symptom network graph based on the causality data.

In some implementations, at least some of the edges between symptom nodes include arrows indicating a direction of causality between respective symptom nodes as indicated by the causality data.

In some implementations, generating the symptom network graph further includes applying the physiological data and the EMA data as input to a clustering model to obtain correlation data indicating correlations between symptoms measured by the physiological data and the EMA data; applying the physiological data and the EMA data as input to an auto-regression model to obtain causality data indicating casuals relationships between symptoms measured by the physiological data and the EMA data; generating, based on the correlation data and the causality data, an adjacency matrix representing relationships between symptoms of the patient's psychopathology; and generating the symptom network graph based on the adjacency matrix.

In some implementations, generating the symptom network graph can further include generating an animated symptom network graph by generating a plurality of symptom network graphs over a series of time intervals, each symptom network graph being generated based on the new physiological data and the EMA data obtained during the time intervals; and assembling the animated symptom network graph by combining the symptom network graphs as a sequence of frames of the animated symptom network graph.

Particular implementations of the subject matter described in this specification can be implemented so as to realize one or more of the following technical advantages. Implementations provide unique data visualization output for clinicians to visualize a patient's psychopathology. For example, implementations generate a user interface that presents a patient's complex psychopathological symptoms in a symptom graph, thereby, permitting visualization of complex psychological conditions.

The details of one or more implementations of the subject matter of this disclosure are set forth in the accompanying drawings and the description below. Other features, aspects, and advantages of the subject matter will become apparent from the description, the drawings, and the claims.

### DESCRIPTION OF DRAWINGS

FIG. 1 depicts a block diagram of an example system for determining the symptom network of a patient's psychopathology.

FIG. 2 depicts exemplary time dependent graphs of various types of patient data (symptom data).

FIG. 3 depicts an exemplary adjacency matrix of a patient's psychopathology symptoms.

FIG. 4 depicts an exemplary symptom network graph for a patient.

FIG. 5 depicts a flowchart of an example process for determining the symptom network of a patient's psychopathology.

FIG. 6 depicts a schematic diagram of a computer system that may be applied to any of the computer-implemented methods and other techniques described herein.

Like reference numbers and designations in the various drawings indicate like elements.

### DETAILED DESCRIPTION

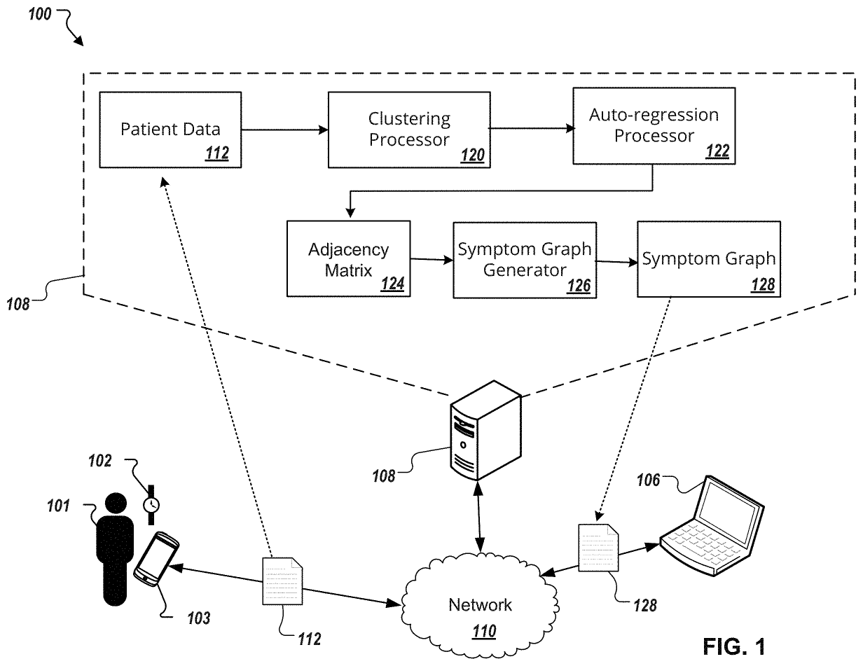

FIG. 1 is a diagram that illustrates an example system 100 for monitoring the evolution of a patient's psychopathology with a symptom network graph. The system 100 includes a psychopathology evaluation system (PES) 108 in communication with a plurality patient computing devices 102, 103, 106 over a network 110. Network 110 can include public and/or private networks and can include the Internet.

The PES 108 can include a system of one or more computers. In general, the PES 108 is configured to perform one or more types of machine learning processes on a combination of time dependent data, e.g., time-dependent psychological and physiological data (collectively patient data 112), to develop a graphical representation of a patient's psychopathology. The PES 108 obtains patient psychological and physiological data over a period of time (e.g., a period of days, weeks, or months). The psychological data can include, but is not limited to, patient ecological momentary assessment (EMA) questions received from to a patient's computing device 103 at intervals throughout the day. The physiological data can include measurements of various patient physiological parameters received from a wearable device 102 that include, but are not limited to, sleep onset latency, sleep duration, wake after sleep onset (WASO), heart rate, heart rate variability, daily step count, or any combination thereof. The PES 108 then applies a series of one or more machine learning algorithms to the combined data to generate an adjacency matrix representation of various psychopathological symptoms that define the patient's individual psychopathology. For example, the PES 108 can store and execute one or more machine learning algorithms such as, e.g., clustering algorithms, auto regression algorithms, or both. The PES 108 processes the adjacency matrix to generate a symptom graph 128 representation of the patient's psychopathology, which can be transmitted as an output to the patient's computing device 103 or a clinician's computing device 106.

In some implementations, the PES 108 generates and stores new adjacency matrices for a patient periodically (e.g., weekly, monthly, etc.). The PES 108 can generate an animated symptom graph 128 depicting changes in the patient's symptom graph 128 overtime. The animated symptom graph 128 may provide clinicians with a unique graphical interface to visualize the evolution of a patient's psychopathology over time. For example, the animated symptom graph 128 may permit clinicians to objectively observe how various treatments and/or medications affect a patient's psychopathology.

In some implementations, data access rules executed by the PES 108 permit the PES 108 to obtain patient data 112 without third-party human interaction with the data on the PES 108, thereby, protecting patient privacy. The PES 108 can further protect each patient's privacy by the PES 108 assigning anonymized patient identifiers to each patient 101 whose data is obtained. The PES 108 can use the anonymized patient identifiers to correlate data to specific patients while protecting personal information. For example, the system can remove personally identifiable information and assign a unique patient identifier to each unique patient. In some examples, the patient identifiers may be non-reversible to protect each patient's identity. In some examples, the system can perform a cryptographic hash function on particular aspects of each patient's identity, e.g., the system can hash a combination of the patient's name, address, and date of birth to obtain a unique patient identifier.

Wearable devices 102 can be wearable computing devices, e.g., smart watches, health tracking devices, smart rings. Computing devices 103, 106 can be computing devices, e.g., mobile phones, smart phones, tablet computers, laptop computers, desktop computers, home assistant devices, or other portable or stationary computing device. Computing device 106 can be a computing device associated with a clinician (e.g., a psychologist or a psychiatrist) to which the PES 108 transmits patient symptom graphs and/or animated symptom graphs 128.

In some implementations, the PES 108 can permit authorized users (e.g., clinicians) to interact with subsets of the psychological data 112 and patient symptom graphs 128. For example, the PES 108 can provide a secure portal for a clinician to select a subset of patient data for analysis by the PES 108. In such implementations, the clinician can be restricted to interacting only with data related to his or her own patients. Thus, a clinician can configure various analyses for particular patients or subsets of patients.

In various implementations, PES 108 can perform some or all of the operations related to generating individualized patient symptom graphs 128. For example, PES 108 can include a cluster processor 120, an auto-regression processor 122, and a symptom graph generator 126. The cluster processor 120, auto-regression processor 122, and symptom graph generator 126 can each be provided as one or more computer executable software modules or hardware modules. That is, some or all of the functions of cluster processor 120, auto-regression processor 122, and symptom graph generator 126 can be provided as a block of code, which upon execution by a processor, causes the processor to perform functions described below. Some or all of the functions of cluster processor 120, auto-regression processor 122, and symptom graph generator 126 can be implemented in electronic circuitry, e.g., as field programmable gate array (FPGA) or an application specific integrated circuit (ASIC).

PES 108 can also implement one or more machine learning algorithms that analyze the patient data 112 to generate symptom graphs 128. For example, clustering processor 120 can be implemented as one or more machine learning models. More specifically, clustering processor 120 can execute one or more clustering models that have been trained to receive model inputs (e.g., anonymized patient data 112) and identify correlations between the different types of data streams in the patient data 112, as described in more detail below. In some implementations, the clustering processor 120 executes a community detection algorithm, a k-mean clustering algorithm, a mean-shift clustering algorithm, an expectation-maximization clustering algorithm, or an agglomerative hierarchical clustering algorithm.

In some examples, auto-regression processor 122 can be implemented as one or more machine learning models. More specifically, auto-regression processor 122 can execute one or more auto-regression models that have been trained to receive model inputs (e.g., anonymized patient data 112) and identify causal correlations between the different types of data streams in the patient data 112, as described in more detail below

In operation, PES 108 collects patient data 112 from a patient's 101 computing device(s) 103 and wearable device(s) 102. More specifically, patient data 112 can include multiple time dependent streams or “channels” of different types of patient physiological and EMA data.

For example, the PES 108 can receive patient physiological data from the patient's 101 wearable device 102 over a period of time (e.g., days, weeks, months). Physiological data can include, but is not limited to, measurements of patient physiological characteristics such as sleep onset latency, sleep duration, wake after sleep onset (WASO), heart rate, heart rate variability, daily step count, or any combination thereof. The PES 108 can receive uploads of physiological data from the wearable device 102 of patient's 101 who have opted-in to the PES 108 analysis, e.g., at the advice or with the assistance of a clinician. The PES 108 can receive regular (e.g., daily) uploads of patient physiological data that includes periodic measurements of the various physiological characteristics noted above.

The PES 108 can also receive patient EMA data, e.g., to determine the state of the patient's 101 psychological health over time. For example, the PES 108 can prompt the patient's computing device 103 to present EMA prompts to the patient at various intervals throughout the day in order to obtain patient responses to the question. EMA prompts can include questions directed to pleasure processing, depressive disorder, or feelings of self-worth, e.g., feelings of hopelessness, feelings of irritability, feelings of fatigue, difficulty concentration, or difficulty making decisions. The PES 108 receives the patient's 101 responses to the prompts at the respective time intervals. For example, an EMA prompt may request that the patient score their feelings of irritability, fatigue, ability to concentrate, etc. on a scale of 1 to 10 at various times throughout the day (e.g., every few hours). The patient 101 can submit EMA responses to the PES 108 by responding to the EMA questions on their computing device 103.

On the whole, the PES 108 can receive multiple channels of both patient physiological data and patient EMA data each day for a period of weeks, months, or years. Moreover, the particular types of physiological data and EMA data collected may differ for each particular patient dependent on the patient's circumstances. A clinician may be permitted to select particular patient data types for processing by the PES 108 for each of his or her patients. For instance, over the course of a day the PES 108 may receive the following types of patient data 112 for a particular patient 101:
1) Measurements of the patient's 101 sleep pattern over the past 24 hours;
2) Measurements of the patient's 101 pulse rate at regular intervals (e.g., every five minutes) over the past 24 hours;
3) Measurements of the patient's 101 step count at regular intervals (e.g., every thirty minutes) over the past 24 hours;
4) Responses to EMA prompts related to concentration at regular intervals (e.g., hourly over the past 24 hours (e.g., waking hours only);
5) Responses to EMA prompts related to irritability at regular intervals (e.g., hourly over the past 24 hours (e.g., waking hours only);
6) Responses to EMA prompts related to decision making ability at regular intervals (e.g., hourly over the past 24 hours (e.g., waking hours only); and
7) Responses to EMA prompts related to feelings of hopelessness at regular intervals (e.g., hourly over the past 24 hours (e.g., waking hours only).
   Furthermore, each of these seven data types may be indicative of at least one symptom that will be represented by the symptom graph 128

The PES 108 applies the patient data 112 as input to the clustering processor 120 and to the auto-regression processor 122. For example, the PES may accumulate patient data 112 over the course of several days or weeks before analyzing the patient data 112 using the clustering processor 120 and to the auto-regression processor 122, e.g., to ensure sufficient data is available to develop a symptom graph 128 for the patient. Once sufficient patient data 112 has been collected for a particular patient 101, the PES 108 can update the analysis of that patient's 101 data at regular intervals (e.g., daily, weekly, or month) by incorporating the data received over the time interval with the patient's past data. For instance, regular analyses of a particular patient can be used to generate the animated symptom graphs noted above.

The clustering processor 120 executes a clustering algorithm to identify correlations between the different types of patient data 112 received. For example, the clustering processor 120 identifies correlations between the different data types based on variations in symptoms observed over time. That is, the clustering processor 120 clusters data based on similar variations between two types of data over time, thus indicative of similar variations between two different symptoms. In addition to identifying correlations between symptoms, the clustering processor 120 can determine correlation strength between symptoms and represent the strength with a numerical score. In some examples, the clustering processor 120 generates correlation data (e.g., correlation scores between the different types of patient data) indicating correlations between different symptoms indicated by the different types of patient data. The correlation data can be used to develop an adjacency matrix indicating correlations between various symptoms of the patient's psychopathology, as discussed below.

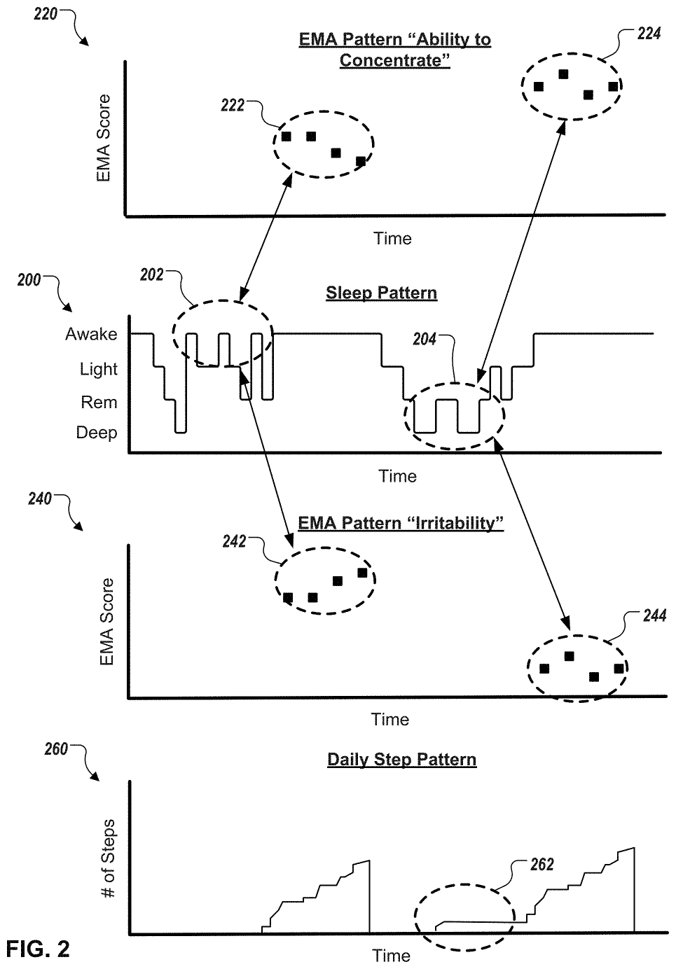

For example, FIG. 2 depicts exemplary time dependent graphs 200, 220, 240 of various types of patient data or symptom data that may be received by the PES 108 and applied to the clustering processor 120. Graphs 200, 220, 240, and 260 represent data received over the course of two days. The data illustrated in the graphs 200, 220, 240, and 260 is simplified for the purpose of explanation. Graph 200 illustrates an example of patient sleep pattern data. Graph 220 illustrates example EMA response scores related to a patient's 101 ability to concentrate. Graph 240 illustrates example EMA response scores related to a patient's 101 irritability. Graph 260 illustrates example measurements of the patient's 101 daily steps accumulated throughout the day and resetting each evening.

Using graphs 200 and 220 as an example, the clustering processor 120 may identify a correlation between the patients sleep pattern and the patient's ability to concentrate; and more specifically between the patient's WASO pattern and ability to concentrate. For example, region 202 shows that the patient woke several times throughout the night and the following day recorded low concentration scores (region 222). Similarly, when the patient does not wake often during the night (region 204), the patient's concentration scores improve (region 224). Clustering processor 120 may also identify a correlation between the patient's WASO pattern and the patient's irritability. For example, graphs 200 and 240 illustrate similar correlations between regions 202 and 242 and regions 204 and 244. That is, the patient's irritability scores are elevated (region 242) following a period of increased WASO during the night (region 202). However, the clustering processor 120 may not identify any correlation between the data in graphs 200, 220, and 240 with the patient's 101 step measurements (graph 260). However, graph 260 may indicate sleep walking (region 262) which may correlate with other symptoms not depicted.

The auto-regression processor 122 executes an auto-regression algorithm to identify causal relationships between the different types of patient data 112 received. For example, the auto-regression processor 122 identifies time dependent causality between correlated symptoms. That is, the auto-regression processor 122 identifies which, if any, symptoms may be causing or contributing to the cause of other symptoms. In some examples, the auto-regression processor 122 generates causality data (e.g., causality scores between the different types of patient data) indicating causal relationships between different symptoms indicated by the different types of patient data. The causality data can be combined with the correlation data to develop a directed graph adjacency matrix, as discussed below.

Causality may not be found between all correlated symptoms. In some implementations, the PES 108 can apply clustered patient data as input to the auto-regression processor 122 to identify causal relationships between the patient's symptoms as clustered by the clustering processor 120. In addition to identifying causality between symptoms, the auto-regression processor 122 can determine causal strength between symptoms and represent the strength with a numerical score. In some implementations, the PES 108 can apply un-clustered patient data 112 as input to the auto-regression processor 122 to identify causal relationships between un-clustered symptoms.

Using graphs 200, 220, and 240 as an example, the auto-regression processor 122 may identify a causal relationship between the patient's WASO pattern (graph 200) and both the ability to concentrate (graph 220) and irritability (graph 240). For example, the auto-regression processor 122 can identity temporal causality between symptoms by identifying changes in one symptom (e.g., decreased concentration region 222 and improved concentration region 224) that occur in response to a corresponding change in another symptom (e.g., increased WASO region 202 and improved WASO 204).

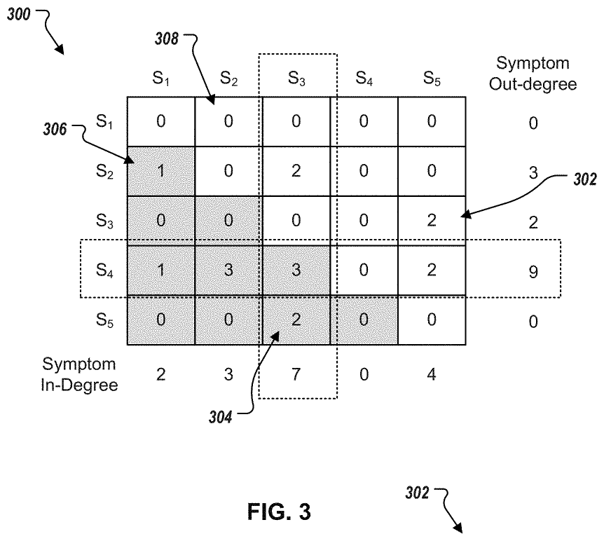

In some implementations, correlation and causality between symptoms can be represented by scores in an adjacency matrix, such as adjacency matrix 300 depicted in FIG. 3. For example, the PES 108 can combine the results of the clustering analysis and the auto-regression analysis into an adjacency matrix 300 representing both correlation and causality between symptoms. Adjacency matrix 300 represents a matrix for a directed graph (e.g., indicating causality). Correlation/Causation strength between symptoms S₁ through S₅ is represented by the magnitude of the scores within the cells of the matrix 300. Correlation without a causal relationship is represented by symmetric entries in the matrix 300. For instance, cells 302 and 304 each include identical correlation scores between symptoms S₃ and S₅ indicating that there is no directivity, e.g., causality, between the symptoms although there is a moderate correlation. Causation is represented by asymmetry between cells in the matrix 300. For instance, cell 306 includes a correlation score indicating a weak correlation from symptom S₂ to symptom S₁, but cell 308 is null indicating that there appears to be “no correlation” from symptom S₁ to symptom S₂. Consequently, the score in cell 306 is a causation score and indicates a directed (e.g., causal) correlation between symptoms S₂ and S₁ in which symptom S₂ is a weak (e.g., a score of 1) cause or contributing cause of symptom S₁.

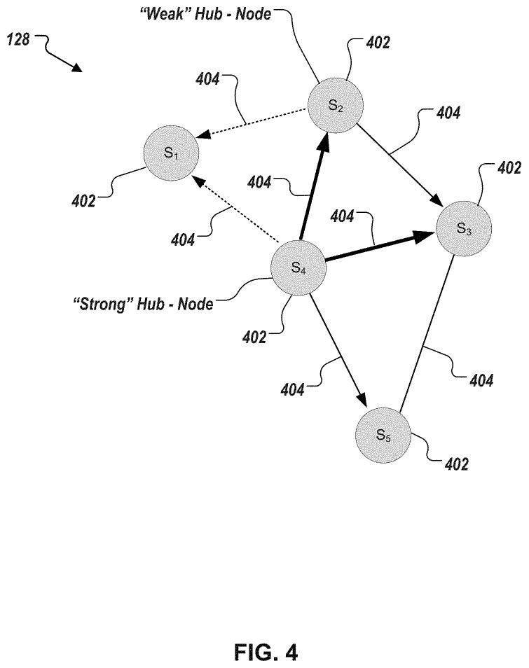

The PES 108 uses the adjacency matrix to generate the symptom graph 128. For example, the symptom graph generator 126 can generate the symptom graph 128 from the adjacency matrix. FIG. 4 illustrates an example symptom graph 128 that may be generated from adjacency matrix 300. The symptom graph 128 represents a patient's idiomatic psychopathological symptom network as determined from their patient data 112 collected over a period of time. The symptom graph 128 includes a plurality of nodes 402 each representing a symptom (S₁-S₅) measured by the patient data 112, and edges 404 representing correlation and causality between symptoms. Edges 404 representing correlation without causation do not include arrows, whereas edges 404 that represent both correlation and causation include an arrow indicating the direction of the causal relationship between symptoms. For example, symptom graph 128 depicts an edge 404 extending between symptom S₄ and S₃ with an arrow indicating a causal relationship from symptom S₄ to symptom S₃. The arrow on this edge 404 indicates that symptom S₄ is a likely cause or contributing cause to symptom S₃.

In some implementations, the relative strength of correlations/causations between symptoms can be represented by a characteristic of the edge 404. Such characteristics can include, but are not limited to, edge thickens, edge color, edge line type. For example, symptom graph 128 uses a thick edge 404 to depict a relatively strong correlation/causation, and a dashed edge 404 to depict a relatively weak correlation/causation. For example, symptoms S₄ and S₃ are correlated with a relatively strong causal correlation indicated by a thick edge 404 with an arrow pointing from S₄ to S₃. Symptoms S₃ and S₅ are correlated with a moderate non-causal correlation as indicated by the medium thickness edge 404 with no arrows extending between S₃ and S₅. And, symptoms S₂ and S₁ are correlated with a relatively weak causal correlation as indicated by a dashed edge 404 with an arrow pointing from S₂ to S₁. In some implementations, numerical correlation/causation scores can be presented above the respective edges 404.

In some implementations, a threshold value can be used to determine which correlation/causation scores are great enough to signify a correlation/causation between two symptoms. For example, symptoms may only be represented by an edge in the symptom graph 128 when a magnitude of the correlation/causation score between the two is greater than or equal to the threshold value.

In some implementations, “hub-nodes” can be identified. A “hub-node” is a graph node 402 such as symptom S₄ which has a relatively large number of edges 404 directed away from the node 402. In other words, a “hub-node” is a likely cause or contributing cause of a relatively large number of other symptoms. Identification of “hub-nodes” may aid clinicians in treating patients because “hub-nodes” may identify core symptoms affecting a patient's mental health, allowing a clinician to focus treatment on the most impactful symptoms. PES 108 can identify “hub-nodes” using a symptom out-degree metric calculated from the adjacency matrix 300. For example, the symptom out-degree metric represents the overall causal impact of a particular symptom on all the other symptoms of the patient's psychopathology network. Symptoms with the greatest symptom out-degree represent “hub-nodes.” Row 308 in FIG. 3 highlights the impact of symptom S₄ on the rest of the patient's psychopathology network.

Another metric, symptom in-degree, can be used to identify symptoms that are most impacted by other symptoms. In other words, these are symptoms that are most impacted by causal relationships with other symptoms, in which the other symptoms are causes or contributing causes to the impacted symptom. Symptom S₃ is an example of such. Symptom S₃ is impacted with moderate and strong causality by two other symptoms (S₂ and S₄). This can be identified by the symptom in-degree of symptom S₃ (shown in row 310 of FIG. 3). Symptom S₃ has the greatest symptom in-degree of all the symptoms, and thus is the most impacted symptom in the patient's psychopathology network.

Once the adjacency matrix 300 and symptom network graph 400 have been generated by the symptom network graph system 126, the adjacency matrix 300 and symptom network graph 400 can be transmitted by the PES 108 over the network 112 to a clinician device 106. The clinician device 106 can be a computing device, e.g., mobile phone, smart phone, tablet computers, laptop computer, desktop computer, home assistant device, or other portable or stationary computing device.

The clinician device 106 can include a program to provide a clinician with multiple outputs respondent to a clinician's decision. A clinician can choose to view part or all of the adjacency matrix 300, part or all of the symptom network graph 400, or a combination thereof.

In some implementations, the PES 108 can generate a new adjacency matrix 300 and symptom graph 128 for each patient on a repeated basis. For example, each week or month the PES 108 can use new patient data 112 received for the patient over that period of time to generate an updated adjacency matrix 300 and symptom graph 128 for the patient. A series of symptom graphs 128 can be generated for each patient over time and combined into an animation that graphically depicts changes in the patient's psychopathology network over time. Such animations may be beneficial to clinicians for evaluating the effects of treatments and/or medications for the patient. For example, clinicians can view the effects of medications and/or treatments on the causal relationships between symptoms and the strength of those relationships as they change over time. In some implementations, the PES 108 may permit clinicians to provide data indicating when a patient begins a particular treatment/medication. For example, the animations can show a clinician how treatment of a “hub-node” symptom affects other symptoms over time. If the “hub-node” symptom diminishes its impact on other symptoms will be illustrated in subsequent frames of the animation. The animations can include annotations (e.g., subtitles) indicating when the treatment/medication began to provide the clinician with context for which treatments and/or medications are being effective.

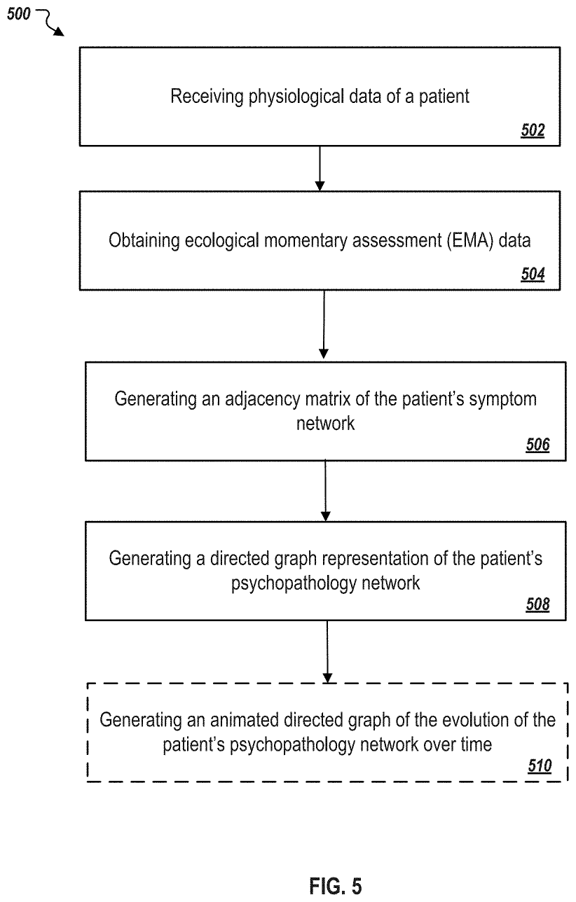

FIG. 5 depicts a flowchart of an example process 500 for tracking an individual patient's psychopathology in accordance with implementations of the present disclosure. In some implementations, the process 500 can be provided as one or more computer-executable programs executed using one or more computing devices such as PES 108. In some examples, process 500 is executed using one or more machine learning models.

The system receives physiological data of a patient over a period of time (502). For example, the system can receive patient physiological data from a wearable device over a period of time (e.g., days, weeks, months). Physiological data can include, but is not limited to, measurements of patient physiological characteristics such as sleep onset latency, sleep duration, wake after sleep onset (WASO), heart rate, heart rate variability, daily step count, or any combination thereof. The system can receive regular (e.g., daily) uploads of patient physiological data that includes periodic measurements of the various physiological characteristics noted above.

The system obtains EMA data of a patient over a period of time (504). For example, the system can transmit EMA prompts to a patient's computing device for presentation to the patient. The system can send EMA prompts at regular intervals or send a set of prompts to the computing device with instructions that cause the computing device to periodically present a prompt to the patient and obtain a response to the prompt. For example, EMA prompts can be presented to the patient at various intervals throughout the day in order to obtain patient responses to the question. EMA prompts can include questions directed to pleasure processing, depressive disorder, or feelings of self-worth, e.g., feelings of hopelessness, feelings of irritability, feelings of fatigue, difficulty concentration, or difficulty making decisions. The system receives the patient's responses to the prompts at the respective time intervals. For example, an EMA prompt may request that the patient score their feelings of irritability, fatigue, ability to concentrate, etc. on a scale of 1 to 10 at various times throughout the day (e.g., every few hours). The patient can submit EMA responses to the system by responding to the EMA questions on their computing device.

The system generates an adjacency matrix for a patient's idiomatic psychopathology symptom network (506). For example, the system generates an adjacency matrix based on the received patient physiological data and EMA responses. The system can generate the adjacency matrix by identifying correlations between different symptoms indicated by the received patient data and causal relationships between the symptoms. For example, the system can apply the patient data as input to a clustering algorithm, an auto-regression algorithm, or both to generate the adjacency matrix. In some implementations, the system can store the adjacency matrix and generate updates to the adjacency matrix as additional patient data is received.

The system generates a directed graph representation of the patient's psychopathology network (e.g., a symptom graph) (508). For example, the system can generate a symptom graph depicting the patient's psychopathology from the adjacency matrix. The system outputs the symptom graph for presentation to a user (e.g., a clinician). The symptom graph is a graphical depiction of a patient's unique psychopathology and depicts relationships between various symptoms of the patient's psychopathology as indicated in the received patient data. For example, the symptom graph can include symptom nodes connected by edges, where the symptom nodes represent individual psychopathological symptoms present in the individual and the edges representing correlations/causality between different symptom nodes as indicated by the patient data. The edges can be determined based on correlation/causation scores between symptoms. Moreover, the magnitude of each correlation/causation score can be represented by characteristics of the edge, e.g., a color, thickness, line type, etc. Causation between symptoms can be represented by arrows on the edges indicating causality or partial causality from one symptom to another. In some implementations, a threshold value can be used to determine which correlation/causation scores are great enough to signify a correlation/causation between symptoms.

In some implementations, the system generates an animated symptom graph of the evolution of the patient's psychopathology network over time. For example, the system can generate a series of symptom graphs for each patient over time and combine the graphs into an animation that graphically depicts changes in the patient's psychopathology network over time. The system can generate a new adjacency matrix and symptom graph for each patient on a repeated basis. For example, each week or month the system can use new patient data 112 received for the patient over that period of time to generate an updated adjacency matrix and symptom graph for the patient. The series of symptom graphs can then be combined as a series of frames within an animation to depict the evolution of the patient's psychopathology over time; permitting a clinician to visualize such changes and potential effects of treatments or medications on the patient's psychopathology.

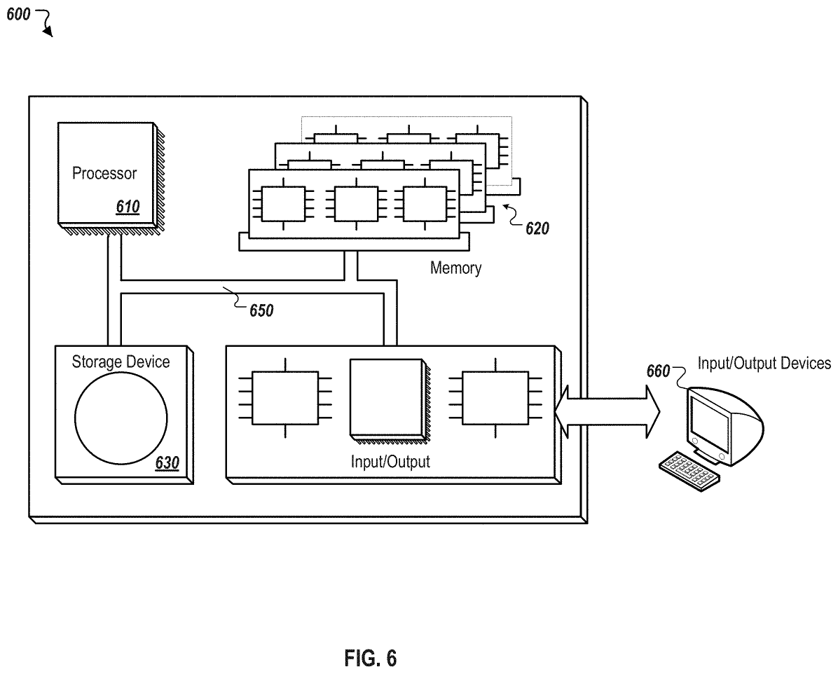

FIG. 6 is a schematic diagram of a computer system 600. The system 600 can be used to carry out the operations described in association with any of the computer-implemented methods described previously, according to some implementations. In some implementations, computing systems and devices and the functional operations described in this specification can be implemented in digital electronic circuitry, in tangibly-embodied computer software or firmware, in computer hardware, including the structures disclosed in this specification (e.g., system 600) and their structural equivalents, or in combinations of one or more of them. The system 600 is intended to include various forms of digital computers, such as laptops, desktops, workstations, personal digital assistants, servers, blade servers, mainframes, and other appropriate computers, including vehicles installed on base units or pod units of modular vehicles. The system 600 can also include mobile devices, such as personal digital assistants, cellular telephones, smartphones, and other similar computing devices. Additionally, the system can include portable storage media, such as, Universal Serial Bus (USB) flash drives. For example, the USB flash drives may store operating systems and other applications. The USB flash drives can include input/output components, such as a wireless transducer or USB connector that may be inserted into a USB port of another computing device.

The system 600 includes a processor 610, a memory 620, a storage device 630, and an input/output device 640. Each of the components 610, 620, 630, and 640 are interconnected using a system bus 650. The processor 610 is capable of processing instructions for execution within the system 600. The processor may be designed using any of a number of architectures. For example, the processor 610 may be a CISC (Complex Instruction Set Computers) processor, a RISC (Reduced Instruction Set Computer) processor, or a MISC (Minimal Instruction Set Computer) processor.

In one implementation, the processor 610 is a single-threaded processor. In another implementation, the processor 610 is a multi-threaded processor. The processor 610 is capable of processing instructions stored in the memory 620 or on the storage device 630 to display graphical information for a user interface on the input/output device 640.

The memory 620 stores information within the system 600. In one implementation, the memory 620 is a computer-readable medium. In one implementation, the memory 620 is a volatile memory unit. In another implementation, the memory 620 is a non-volatile memory unit.

The storage device 630 is capable of providing mass storage for the system 600. In one implementation, the storage device 630 is a computer-readable medium. In various different implementations, the storage device 630 may be a floppy disk device, a hard disk device, an optical disk device, or a tape device.

The input/output device 640 provides input/output operations for the system 600. In one implementation, the input/output device 640 includes a keyboard and/or pointing device. In another implementation, the input/output device 640 includes a display unit for displaying graphical user interfaces.

The features described can be implemented in digital electronic circuitry, or in computer hardware, firmware, software, or in combinations of them. The apparatus can be implemented in a computer program product tangibly embodied in an information carrier, e.g., in a machine-readable storage device for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer. Additionally, such activities can be implemented via touchscreen flat-panel displays and other appropriate mechanisms.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include a local area network (“LAN”), a wide area network (“WAN”), peer-to-peer networks (having ad-hoc or static members), grid computing infrastructures, and the Internet.

The computer system can include clients and servers. A client and server are generally remote from each other and typically interact through a network, such as the described one. The relationship of client and server arises by virtue of computer programs running on the respective computers and having a client-server relationship to each other.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

For convenience, implementations of the present disclosure have been discussed in further detail with reference to an example medical context. More specifically, the example context includes predicting the spread of a contagion (e.g., an illness). It is appreciated, however, that implementations of the present disclosure can be realized in other appropriate contexts (e.g., predicting the spread of ideas, social trends, word-of-mouth advertising, etc.).

What is claimed is:

1. A computer-implemented method executed by one or more, the method comprising:
receiving, from a wearable computing device, physiological data of a patient, the physiological data being received over a period of time;
obtaining, at predetermined time intervals within the period of time from the patient, ecological momentary assessment (EMA) data by, at each time interval:
sending, for presentation on a computing device associated with the patient, an EMA data prompt, and
receiving, from the computing device, patient input responsive to the EMA data prompt; and
generating, based on the EMA data and the physiological data, a graphical representation of the patient's idiomatic psychopathology symptom network as a symptom network graph, the graph comprising symptom nodes connected by edges, the symptom nodes representing individual psychopathologic symptoms present in the individual and the edges representing correlations between different symptom nodes indicated by the EMA data and the physiological data.

2. The method of claim 1, wherein a characteristic of the edges represents strength of correlation between symptom nodes.

3. The method of claim 1, wherein generating the symptom network graph comprises:
applying the physiological data and the EMA data as input to a clustering model to obtain correlation data indicating correlations between symptoms measured by the physiological data and the EMA data; and
generating the symptom network graph based on the correlation data.

4. The method of claim 3, wherein the clustering model is one of a community detection algorithm, a k-mean clustering algorithm, a mean-shift clustering algorithm, an expectation-maximization clustering algorithm, or an agglomerative hierarchical clustering algorithm.

5. The method of claim 1, wherein generating the symptom network graph comprises:
applying the physiological data and the EMA data as input to an auto-regression model to obtain causality data indicating casuals relationships between symptoms measured by the physiological data and the EMA data; and
generating the symptom network graph based on the causality data.

6. The method of claim 5, wherein at least some of the edges between symptom nodes include arrows indicating a direction of causality between respective symptom nodes as indicated by the causality data.

7. The method of claim 1, wherein generating the symptom network graph comprises:
applying the physiological data and the EMA data as input to a clustering model to obtain correlation data indicating correlations between symptoms measured by the physiological data and the EMA data;
applying the physiological data and the EMA data as input to an auto-regression model to obtain causality data indicating casuals relationships between symptoms measured by the physiological data and the EMA data;
generating, based on the correlation data and the causality data, an adjacency matrix representing relationships between symptoms of the patient's psychopathology; and
generating the symptom network graph based on the adjacency matrix.

8. The method of claim 1, further comprising generating an animated symptom network graph by:
generating a plurality of symptom network graphs over a series of time intervals, each symptom network graph being generated based on the new physiological data and the EMA data obtained during the time intervals; and
assembling the animated symptom network graph by combining the symptom network graphs as a sequence of frames of the animated symptom network graph.

9. A system comprising:
at least one processor; and a data store coupled to the at least one processor having instructions stored thereon which, when executed by the at least one processor, causes the at least one processor to perform operations comprising:
receiving, from a wearable computing device, physiological data of a patient, the physiological data being received over a period of time;
obtaining, at predetermined time intervals within the period of time from the patient, ecological momentary assessment (EMA) data by, at each time interval:
sending, for presentation on a computing device associated with the patient, an EMA data prompt, and
receiving, from the computing device, patient input responsive to the EMA data prompt; and
generating, based on the EMA data and the physiological data, a graphical representation of the patient's idiomatic psychopathology symptom network as a symptom network graph, the graph comprising symptom nodes connected by edges, the symptom nodes representing individual psychopathologic symptoms present in the individual and the edges representing correlations between different symptom nodes indicated by the EMA data and the physiological data.

10. The system of claim 9, wherein generating the symptom network graph comprises:
applying the physiological data and the EMA data as input to a clustering model to obtain correlation data indicating correlations between symptoms measured by the physiological data and the EMA data; and
generating the symptom network graph based on the correlation data.

11. The system of claim 10, wherein the clustering model is one of a community detection algorithm, a k-mean clustering algorithm, a mean-shift clustering algorithm, an expectation-maximization clustering algorithm, or an agglomerative hierarchical clustering algorithm.

12. The system of claim 9, wherein generating the symptom network graph comprises:
applying the physiological data and the EMA data as input to an auto-regression model to obtain causality data indicating casuals relationships between symptoms measured by the physiological data and the EMA data; and
generating the symptom network graph based on the causality data.

13. The system of claim 12, wherein at least some of the edges between symptom nodes include arrows indicating a direction of causality between respective symptom nodes as indicated by the causality data.

14. The system of claim 9, wherein generating the symptom network graph comprises:
applying the physiological data and the EMA data as input to a clustering model to obtain correlation data indicating correlations between symptoms measured by the physiological data and the EMA data;
applying the physiological data and the EMA data as input to an auto-regression model to obtain causality data indicating casuals relationships between symptoms measured by the physiological data and the EMA data;
generating, based on the correlation data and the causality data, an adjacency matrix representing relationships between symptoms of the patient's psychopathology; and
generating the symptom network graph based on the adjacency matrix.

15. A non-transitory computer readable storage device storing instructions that, when executed by at least one processor, cause the at least one processor to perform operations comprising:
receiving, from a wearable computing device, physiological data of a patient, the physiological data being received over a period of time;
obtaining, at predetermined time intervals within the period of time from the patient, ecological momentary assessment (EMA) data by, at each time interval:
sending, for presentation on a computing device associated with the patient, an EMA data prompt, and
receiving, from the computing device, patient input responsive to the EMA data prompt; and
generating, based on the EMA data and the physiological data, a graphical representation of the patient's idiomatic psychopathology symptom network as a symptom network graph, the graph comprising symptom nodes connected by edges, the symptom nodes representing individual psychopathologic symptoms present in the individual and the edges representing correlations between different symptom nodes indicated by the EMA data and the physiological data.

16. The device of claim 15, wherein generating the symptom network graph comprises:
applying the physiological data and the EMA data as input to a clustering model to obtain correlation data indicating correlations between symptoms measured by the physiological data and the EMA data; and
generating the symptom network graph based on the correlation data.

17. The device of claim 16, wherein the clustering model is one of a community detection algorithm, a k-mean clustering algorithm, a mean-shift clustering algorithm, an expectation-maximization clustering algorithm, or an agglomerative hierarchical clustering algorithm.

18. The device of claim 15, wherein generating the symptom network graph comprises:
applying the physiological data and the EMA data as input to an auto-regression model to obtain causality data indicating casuals relationships between symptoms measured by the physiological data and the EMA data; and
generating the symptom network graph based on the causality data.

19. The device of claim 18, wherein at least some of the edges between symptom nodes include arrows indicating a direction of causality between respective symptom nodes as indicated by the causality data.

20. The device of claim 15, wherein generating the symptom network graph comprises:
applying the physiological data and the EMA data as input to a clustering model to obtain correlation data indicating correlations between symptoms measured by the physiological data and the EMA data;
applying the physiological data and the EMA data as input to an auto-regression model to obtain causality data indicating casuals relationships between symptoms measured by the physiological data and the EMA data;
generating, based on the correlation data and the causality data, an adjacency matrix representing relationships between symptoms of the patient's psychopathology; and
generating the symptom network graph based on the adjacency matrix.
